SEQUENCE LISTING (5 sequences)

SEQ ID NO: 1 - DNA, 30 bp, Artificial Sequence
  random synthetic sequence
ctccctaataacaatttataactattccta

SEQ ID NO: 2 - DNA, 30 bp, Artificial Sequence
  random synthetic sequence
taggaatagttataaattgttattagggag

SEQ ID NO: 3 - DNA, 15 bp, Artificial Sequence
  random synthetic sequence
cgttaggacttacgc

SEQ ID NO: 4 - DNA, 15 bp, Artificial Sequence
  random synthetic sequence
ttataactattccta

SEQ ID NO: 5 - DNA, 30 bp, Artificial Sequence
  random synthetic sequence
taggaatagttataagcgtaagtcctaacg

(19) United States
(12) Patent Application Publication
(10) Pub. No.: US 2009/0155785 A1
(43) Pub. Date: Jun. 18, 2009

(54) REAL-TIME COLORIMETRIC SCREENING INHIBITORS OF ENDONUCLEASE WITH GOLD NANOPARTICLE SUBSTRATE

(75) Inventors: Chad A. Mirkin, Wilmette, IL (US); Xiaoyang Xu, Evanston, IL (US); Min Su Han, Kyung Ju (KR)

Correspondence Address: GREGORY T. PLETTA;Nanosphere, Inc., 4088 Commerical Avenue, Northbrook, IL, 60062-1829, US

(73) Assignee: NORTHWESTERN UNIVERSITY, Evanston, IL (US)

(21) Appl. No.: 12/020,506

(22) Filed: Jan. 25, 2008

Related U.S. Application Data
(60) Provisional application No. 60/897,705, filed on Jan. 25, 2007.

(51) Int. Cl. C12Q 1/68

(52) U.S. Cl. 435/6; 977/904

(57) ABSTRACT

The invention provides methods for screening a compound for its effect on endonuclease activity. The methods comprise providing a compound to be screened utilizing a gold nanoparticle aggregate as the substrate for the endonuclease. The gold nanoparticle aggregate is formed by the hybridization of oligonucleotides attached to the nanoparticles, with or without the presence of a third linker oligonucleotide. The hybridized oligonucleotide duplex serves as a substrate for the endonuclease. A detectable change is brought about in the presence of the endonuclease activity. A decrease in the detectable change reflects the reduced levels of endonuclease activities as a result of the effects of endonuclease inhibitors. The present invention also provides kits for screening an endonuclease inhibitor.

### CROSS REFERENCE TO RELATED APPLICATION

The application claims the benefit of priority to U.S. provisional application Ser. No. 60/897,705, filed Jan. 25, 2007, the disclosure of which is incorporated hereby by reference in its entirety.

The invention was funded by NSF/NSEC Grant No. EEC-0118025, Air Force Office of Science Research (AFOSR) Grant No. F49620-01-1-0401. The Government has certain rights in the invention.

### FIELD OF THE INVENTION

The present invention relates to the fields of enzymology, molecular biology, and diagnostic assay. In particular, the present invention relates to methods, compositions and kits for high throughput screening of an endonuclease inhibitor using functionalized gold nanoparticles.

### BACKGROUND OF THE INVENTION

Nucleic acids act as the carriers of genetic information for all known organisms, and all organisms contain a type of enzymes, called nucleases, (see E. S. Rangarajan, V. Shankar, FEMS Microbiol. Rev., 25: 583 (2001)) which hydrolyze the phosphodiester linkages in the nucleic acids backbone. These nucleases are important for many processes involving replication, repair, and recombination. Nucleases can be categorized into two families: endonucleases and exonucleases. Endonucleases such as DNA gyrase and virus integrase play key roles in biological process such as topological altering of DNA and the insertion of proviral DNA into host chromosomal DNA. (see L. A. Mitscher, Chem. Rev., 105: 559 (2005); H. Zhao, N. Neamati, S. Sunder, H. Hong, S. Wang, G. W. A. Milne, Y. Pommier, T. R. Burke, Jr., J. Med. Chem., 40: 937 (1997)). Molecules that inhibit endonucleases are therefore considered candidates for a variety of anti-microbial and anti-viral drugs. To develop efficient enzyme inhibitors, it is essential to be able to evaluate the activity of the target enzymes in the presence of different inhibitors.

Combinatorial libraries of potential pharmaceutical candidates and high-throughput screening strategies have become a necessary part of drug development. (see P. A. Johnston, P. A. Johnston, Drug Discovery Today, 7: 353 (2002); D. L. Boger, J. Deshamais, K. Capps, Angew. Chem. Int. Ed., 42: 4138 (2003); S. Wang, T. B. Sim, Y.-S. Kim, Y.-T. Chang, Cum Opin. Cham. Biol., 8: 371 (2004)). The most commonly employed assays are those that produce a spectrophotometric signal using simple reagents, in particular chromogenic or fluorogenic substrates. (see E. D. Matayoshi, G. T. Wang, G. A. Krafft, J. Erickson Science, 247: 954 (1990); J.-L. Reyrnond, D. Wahler, Chem Bio Chem, 3: 701 (2002)). In many cases, however, it is desirable to measure the reaction between an enzyme and a well-defined substrate of interest as opposed to a fluorogenic or chromogenic derivative of that substrate. Historically, the endonuclease activity has been screened by viscometry, radioactive labeling, and gel electrophoresis techniques, in addition to the more recent fluorescence-based approaches. (see M. Laskowski, M. D. Seidel Arch. Biochem., 7: 465 (1945); E. P. Geiduschek, A. Daniels, Anal. Biochem., 11: 133 (1965); R. Kohen, M. Szyf, M. Chevion, Anal. Biochem., 154: 455 (1986)). These protocols are time consuming and do not provide a measure of endonuclease activity in real time. Of these methods, only fluorescence is appreciably used for high-throughput screening, and the fluorescence-based approach has only recently been implemented. (see R. Eisenschmidt, T. Lanio, A. Jeltsch, A. Pingoud, J. Biotechnol., 96: 185 (2002)). However, none of the methods is sufficiently sensitive and have several disadvantages when used in high-throughput screening. Thus, a more sensitive method is needed.

### SUMMARY OF THE INVENTION

In one aspect, the invention provides a method for screening one or more types of compounds for their effect on an activity of one or more types of endonucleases, said method comprising steps of: contacting one or more types of gold nanoparticle aggregate substrates with one or more types of endonucleases, in the presence or absence of one or more types of compounds to form at least one reaction mixture and incubating the reaction mixture under conditions sufficient to allow the endonuclease reaction to occur, wherein the gold nanoparticle aggregate substrate comprises at least two types of gold nanoparticles, the first type of gold nanoparticle having one or more first oligonucleotides bound thereto, and the second type of gold nanoparticle having one or more second oligonucleotides bound thereto, the first oligonucleotide and the second oligonucleotide having sequences that are at least partially complementary to each other, wherein the gold nanoparticle aggregate is formed by hybridization of the first and second oligonucleotides, and each type of gold nanoparticle aggregate substrate comprises gold nanoparticles of a unique particle size; and determining the effect of the compound on the endonuclease activity by observing a detectable change between the reaction mixture in the presence of the compound and the reaction mixture in the absence of the compound, wherein a detectable change indicates that the compound has an effect on the endonuclease activity.

In another aspect, the invention provides a method for screening one or more compounds for their effect on an activity of one or more endonucleases, said method comprising steps of: contacting one or more gold nanoparticle aggregate substrates and one or more endonucleases in the presence or absence of one or more compounds to form at least one reaction mixture and incubating the reaction mixture under conditions sufficient to allow the endonuclease reaction to occur, wherein the gold nanoparticle aggregate substrate comprises (1) at least two types of gold nanoparticles, the first type of gold nanoparticle having one or more first oligonucleotides bound thereto, and the second type of gold nanoparticle having one or more second oligonucleotides bound thereto, and (2) at least one type of linker oligonucleotide, the first oligonucleotide and the second oligonucleotide having sequences that are at least partially complementary to the sequence of the linker oligonucleotide, and wherein the gold nanoparticle aggregate is formed by hybridization of the first and second oligonucleotides to the linker oligonucleotide and each type of gold nanoparticle aggregate substrate comprises gold nanoparticles of a unique particle size; and determining the effect of the compound on the endonuclease activity by observing a detectable change between the reaction mixture in the presence of the compound and the reaction mixture in the absence of the compound, wherein a detectable change indicates that the compound has an effect on the endonuclease activity.

In one embodiment of this aspect, the endonuclease is a sequence specific endonuclease. In another embodiment, the endonuclease is a non-sequence specific endonuclease.

In yet another embodiment, the gold nanoparticle aggregate substrate, the endonuclease, and the compound are added simultaneously to form a reaction mixture. In a further embodiment, the gold nanoparticle aggregate substrate is contacted with the compound to form a first reaction mixture, and the endonuclease is contacted with the first reaction mixture to form a second reaction mixture, and wherein the second reaction mixture is incubated under conditions sufficient to allow the endonuclease reaction to occur. In yet another embodiment, the endonuclease is contacted with the compound to form a first reaction mixture, and the gold nanoparticle aggregate is contacted with the first reaction mixture to form a second reaction mixture, and wherein the second reaction mixture is incubated under conditions sufficient to allow the endonuclease reaction to occur.

In another embodiment, the gold nanoparticle has a diameter ranging from about 5 nm to about 250 nm. In a further embodiment, the gold nanoparticle has a diameter of 13 nm.

In yet another embodiment, the method further comprises: providing a plurality of compounds to be screened; contacting the gold nanoparticle aggregate substrate, the endonuclease and the plurality of compounds to form a plurality of reaction mixtures and incubating the plurality of reaction mixtures under conditions sufficient to allow the endonuclease reaction to occur; and determining the effect of any one of the plurality of compounds on the endonuclease activity by observing a detectable change.

In a further embodiment, the gold nanoparticle aggregate substrate, the endonuclease, and each of the plurality of compounds are added simultaneously to form a plurality of reaction mixtures.

In another embodiment, the gold nanoparticle aggregate substrate is contacted with each of the plurality of compounds to form a plurality of first reaction mixtures, and the endonuclease is contacted with each of the first reaction mixture to form a plurality of second reaction mixtures, and wherein each of the second reaction mixture is incubated under conditions sufficient to allow the endonuclease reaction to occur.

In yet another embodiment, the endonuclease is contacted with each of the plurality of compounds to form a plurality of first reaction mixtures, and the gold nanoparticle aggregate substrate is contacted with each of the first reaction mixture to form a plurality of second reaction mixtures, and wherein each of the second reaction mixture is incubated under conditions sufficient to allow the endonuclease reaction to occur.

In a further aspect, the invention provides a kit for screening for an endonuclease inhibitor comprising: (a) a composition comprising a gold nanoparticle aggregate; (b) an endonuclease enzyme; (c) a buffer; and optionally (d) an instruction manual. In one embodiment, the endonuclease enzyme is a lyophilized endonuclease enzyme.

### BRIEF DESCRIPTION OF THE DRAWINGS

FIG. 1 shows a schematic illustration depicting that aggregates made from DNA functionalized gold nanoparticles can be used to screen endonuclease activity and inhibition. Nanoparticle aggregates formed from duplex DNA interconnects are bluish-purple due to the distance-dependent plasmonic properties of the particles from which they are formed. DNase I cleaves the oligonucleotide duplexes, releasing the AuNPs from the aggregate and effecting a purple to red color change. The time needed to hydrolyse the duplex DNA and thus disrupt the aggregate (T_{H}) is dependent on the potency of the inhibiting ability of the endonuclease inhibitors tested. Therefore, measuring T_{H} provides a rapid and simple way to evaluate and screen potential endonuclease inhibitors.

FIG. 2 shows a schematic illustration representing the aggregation and dissociation pathway of the gold nanoparticle probes used in the calorimetric screening of endonuclease inhibitors. The aggregates remain blue longer in the presence of more potent endonuclease inhibitors than less potent inhibitors.

FIG. 3 shows a schematic illustration of DNA functionalized gold nanoparticle aggregate substrate. Nanoparticle aggregate was formed by ligating the two oligonucleotides that are hybridized to a linker oligonucleotide. The aggregate formation resulted in a red-to-blue color change. A blue-to-red color shift can be observed when DNase I cleaves the oligonucleotide duplex, releasing AuNPs from the aggregate. The time needed to disrupt the aggregate depends on the potency of the endonuclease inhibitors. The high throughput colorimetric assay can be applied for measuring endonuclease activities and determining the relative inhibitory potencies of endonuclease inhibitors.

FIG. 4 is a schematic illustration showing the aggregation and dissociation pathway of the gold nanoparticle aggregate substrates as described in FIG. 3 for the calorimetric screening of endonuclease inhibitors.

FIG. 5 shows normalized dissociation curves for the aggregated gold nanoparticles at different DNase I concentrations. The change in extinction was monitored at 520 nm.

FIG. 6 shows normalized dissociation curves for the aggregated gold nanoparticles, in the absence and presence of endonuclease inhibitors, with the concentration of DNase I at 15 units/ml. The change in the extinction was monitored at 520 nm.

FIG. 7 depicts visible color change as a result of dissociation of gold nanoparticle aggregates in the absence or presence of endonuclease inhibitors (1—control; 2—AMSA; 3—AQ2A; 4—9-AA; 5—EIPT; 6—DNR; 7—EB; 8—DAPI) at specific times after adding DNase I.

FIG. 8 shows normalized dissociation curves of the aggregated gold nanoparticle substrates with different DNase I concentration ranging from 10 units/ml to 50 units/ml (in the absence of any endonuclease inhibitors). The change in the extinction was monitored at 520 nm.

FIG. 9 shows normalized dissociation curves of the aggregated gold nanoparticle substrates in the absence and presence of endonuclease inhibitors with the concentration of DNase I at 15 units/ml. The change in the extinction was monitored at 520 nm.

FIG. 10 shows the color change as a result of dissociation of the gold nanoparticle aggregate substrates in the absence (1—control) and presence of endonuclease inhibitors (2—AMSA; 3—AQ2A; 4—9-AA; 5—EIPT; 6—DNR; 7—EB; 8—DAPI) at specific time after adding DNase I.

### DETAILED DESCRIPTION OF THE INVENTION

The current invention provides methods for screening a compound for its effect on endonuclease activity. We report a method for the real-time colorimetric screening of endonuclease inhibitors based upon oligonucleotide functionalized gold nanoparticle aggregates (DNA-AuNPs). This is the first example of using DNA-AuNPs as colorimetric screening modalities for nuclease activity and inhibition. This method takes advantage of the high optical cross section of gold nanoparticles and their distance-dependent property due to through-space interactions between the nanoparticles. In this assay, nanoparticle aggregates were formed by hybridizing two sets of AuNPs. A blue-to-red color change can be observed upon DNase I cleavage of the oligonucleotides, which results in the release of individual AuNPs from the aggregate. The time needed to hydrolyze 50% of the aggregates, T_{H}, depends on the properties of a specific nuclease. For a given nuclease, T_{H} depends on the potency of the nuclease inhibitors tested. Therefore, this assay provides a fast, simple, and sensitive method for screening nuclease and nuclease-inhibitor activity.

### DEFINITIONS

The term “sample” as used herein, is used in its broadest sense and includes, without limitation, a pure, partially purified or crude enzyme or mixtures of enzymes, cell lysates, subcellular fractions, bodily fluid, or tissue homogenates.

The term “detection” as used herein, refers to quantitatively or qualitatively determining the effect of a test compound on the endonuclease. The term “detection” as used herein also refers to determining the presence or absence of an inhibitor in a sample.

The term “compound” as used herein, refers to a test compound, i.e., a compound whose effects on an enzyme activity such as an endonuclease activity are to be ascertained. The compound can be, without limitation, an inhibitor or an activator of the endonuclease activity. The term “compound” as used herein does not refer to a reagent that is an integral component of an enzymatic reaction such as an endonuclease reaction.

The term “substrate” as used herein refers to a reagent, a molecule, a conjugated molecule, or a composition that is acted upon by an enzyme. Likewise, the term “nanoparticle aggregate substrate” as used herein refers to an aggregate of nanoparticles interconnected to one another by at least partially double-stranded oligonucleotides, which a nuclease can cleave.

The term “oligonucleotide” as used herein refers to DNA as well as RNA oligonucleotide. The term “oligonucleotide” may be used interchangeably with nucleic acid.

The term “linker oligonucleotide” as used herein refers to an oligonucleotide that is not attached to a nanoparticle, and that serves to link two or more nanoparticles together by oligonucleotide hybridization.

The term “endonuclease inhibitor” as used herein refers to a reagent, molecule, or compound that decreases the activity of an endonuclease. The inhibitor can be a competitive or a non-competitive inhibitor. The inhibition of the enzyme by the inhibitor can be reversible or irreversible. Some of the DNA binding molecules as described herein or generally known in the art are suitable endonuclease inhibitors for use in the current invention.

The term “high-throughput screening” as used herein refers to a method for experimentation and screening that allows one to quickly conduct hundreds or thousands of biological, chemical, biochemical or diagnostic tests. Robotics, data processing and control software and sample handling devices, etc. are particularly suitable for high-throughput screening.

As used herein, a “type of” oligonucleotides or nanoparticles having oligonucleotides attached thereto refers to a plurality of that item. “Nanoparticles having oligonucleotides attached thereto” are also sometimes referred to as “nanoparticle-oligonucleotide conjugates” or “nanoparticle functionalized with oligonucleotides.”

### Manufacture of Nanoparticles

The current invention provides a method for screening a compound for its effect on endonuclease activity, said method employing nanoparticle aggregates as an indicator of the levels of the endonuclease activity. Nanoparticles useful in the practice of the invention include metal (e.g., gold, silver, copper and platinum), semiconductor (e.g., CdSe, CdS, and CdS or CdSe coated with ZnS) and magnetic (e.g., ferromagnetite) colloidal materials. Other nanoparticles useful in the practice of the invention include ZnS, ZnO, TiO₂, AgI, AgBr, HgI₂, PbS, PbSe, ZnTe, CdTe, In₂S₃, In₂Se₃, Cd₃P₂, Cd₃As₂, InAs, and GaAs. Nanoparticles with diameters ranging from about 5 nm to about 250 nm (mean diameter), especially from about 5 to about 50 nm, and most especially from about 10 to about 30 nm are particularly suitable for use the current invention. The nanoparticles may also be rods. Gold nanoparticles are particularly suitable for use in the current invention.

Methods of making metal, semiconductor and magnetic nanoparticles are well-known in the art. See, e.g., Schmid, G. (ed.) Clusters and Colloids (VCH, Weinheim, 1994); Hayat, M. A. (ed.) Colloidal Gold: Principles, Methods, and Applications (Academic Press, San Diego, 1991); Massart, R., IEEE Transactions On Magnetics, 17: 1247 (1981); Ahmadi, T. S. et al., Science, 272: 1924 (1996); Henglein, A. et al., J. Phys. Chem., 99: 14129 (1995); Curtis, A. C., et al., Angew. Chem. Int. Ed. Engl., 27: 1530 (1988).

Methods of making ZnS, ZnO, TiO₂, AgI, AgBr, HgI₂, PbS, PbSe, ZnTe, CdTe, In₂S₃, In₂Se₃, Cd₃P₂, Cd₃As₂, InAs, and GaAs nanoparticles are also known in the art. See, e.g., Weller, Angew. Chem. Int. Ed. Engl., 32: 41 (1993); Henglein, Top. Curr. Chem., 143: 113 (1988); Henglein, Chem. Rev., 89: 1861 (1989); Brus, Appl. Phys. A., 53: 465 (1991); Bahncmann, in Photochemical Conversion and Storage of Solar Energy (eds. Pelizetti and Schiavello 1991), page 251; Wang and Herron, J. Phys. Chem., 95: 525 (1991); Olshavsky et al., J. Am. Chem. Soc., 112: 9438 (1990); Ushida et al., J. Phys. Chem., 95: 5382 (1992).

Suitable nanoparticles are also commercially available from, e.g., Ted Pella, Inc. (gold), Amersham Corporation (gold) and Nanoprobes, Inc. (gold).

Gold colloidal particles have high extinction coefficients for the bands that give rise to their beautiful colors. These intense colors change with particle size, concentration, interparticle distance, and extent of aggregation and shape (geometry) of the aggregates, making these materials particularly attractive for colorimetric assays. For instance, hybridization of oligonucleotides attached to gold nanoparticles with oligonucleotides or oligonucleotides attached to other gold nanoparticles results in an immediate color change visible to the naked eye (see, e.g., the Examples).

The nanoparticles, the oligonucleotides or both are functionalized in order to attach the oligonucleotides to the nanoparticles. Such methods are known in the art. For instance, oligonucleotides functionalized with alkanethiols at their 3′-termini or 5′-termini readily attach to gold nanoparticles. See Whitesides, Proceedings of the Robert A. Welch Foundation 39th Conference On Chemical Research Nanophase Chemistry, Houston, Tex., pages 109-121 (1995). See also, Mucic et al. Chem. Commun. 555-557 (1996) (describes a method of attaching 3′ thiol DNA to flat gold surfaces; this method can be used to attach oligonucleotides to nanoparticles). The alkanethiol method can also be used to attach oligonucleotides to other metal, semiconductor and magnetic colloids and to the other nanoparticles listed above. Other functional groups for attaching oligonucleotides to solid surfaces include phosphorothioate groups (see, e.g., U.S. Pat. No. 5,472,881 for the binding of oligonucleotide-phosphorothioates to gold surfaces), substituted alkylsiloxanes (see, e.g. Burwell, Chemical Technology, 4: 370-377 (1974) and Matteucci and Caruthers, J. Am. Chem. Soc., 103: 3185-3191 (1981) for binding of oligonucleotides to silica and glass surfaces, and Grabar et al., Anal. Chem., 67: 735-743 for binding of aminoalkylsiloxanes and for similar binding of mercaptoaklylsiloxanes). Oligonucleotides terminated with a 5′ thionucleoside or a 3′ thionucleoside may also be used for attaching oligonucleotides to solid surfaces. The following references describe other methods which may be employed to attached oligonucleotides to nanoparticles: Nuzzo et al., J. Am. Chem. Soc., 109: 2358 (1987) (disulfides on gold); Allara and Nuzzo, Langmuir, 1: 45 (1985) (carboxylic acids on aluminum); Allara and Tompkins, J. Colloid Interface Sci., 49: 410-421 (1974) (carboxylic acids on copper); Iler, The Chemistry Of Silica, Chapter 6, (Wiley 1979) (carboxylic acids on silica); Timmons and Zisman, J. Phys. Chem., 69: 984-990 (1965) (carboxylic acids on platinum); Soriaga and Hubbard, J. Am. Chem. Soc., 104: 3937 (1982) (aromatic ring compounds on platinum); Hubbard, Acc. Chem. Res., 13: 177 (1980) (sulfolanes, sulfoxides and other functionalized solvents on platinum); Hickman et al., J. Am. Chem. Soc., 111: 7271 (1989) (isonitriles on platinum); Maoz and Sagiv, Langmuir, 3: 1045 (1987) (silanes on silica); Maoz and Sagiv, Langmuir, 3: 1034 (1987) (silanes on silica); Wasserman et al., Langmuir, 5: 1074 (1989) (silanes on silica); Eltekova and Eltekov, Langmuir, 3: 951 (1987) (aromatic carboxylic acids, aldehydes, alcohols and methoxy groups on titanium dioxide and silica); Lee et al., J. Phys. Chem., 92: 2597 (1988) (rigid phosphates on metals).

Each nanoparticle will have a plurality of oligonucleotides attached to it. As a result, each nanoparticle-oligonucleotide conjugate can bind to a plurality of oligonucleotides or nucleic acids having the complementary sequence.

Functionalizing Nanoparticles with Oligonucleotides

Oligonucleotides of defined sequences are used for a variety of purposes in the practice of the invention. Methods of making oligonucleotides of a predetermined sequence are well-known. See, e.g., Sambrook et al., Molecular Cloning: A Laboratory Manual (2nd ed. 1989) and F. Eckstein (ed.) Oligonucleotides and Analogues, 1st Ed. (Oxford University Press, New York, 1991). Solid-phase synthesis methods are particularly suitable for both oligoribonucleotides and oligodeoxyribonucleotides (the well-known methods of synthesizing DNA are also useful for synthesizing RNA). Oligoribonucleotides and oligodeoxyribonucleotides can also be prepared enzymatically.

An oligonucleotide can be attached to a nanoparticle either by its 3′ OH group or a 5′ PO₄³⁻ group through a functional group, such as an SH group.

Any suitable method for attaching oligonucleotides onto the particle, nanoparticle, or nanosphere surface may be used. A particularly suitable method for attaching oligonucleotides onto a surface is based on an aging process described in U.S. Pat. Nos. 6,361,944; 6,506,564; 6,767,702; 6,750,016; U.S. patent application Ser. No. 09/927,777, filed Aug. 10, 2001; and in International Application Nos. PCT/US97/12783, filed Jul. 21, 1997; PCT/US00/17507, filed Jun. 26, 2000; PCT/US01/01190, filed Jan. 12, 2001; PCT/US01/10071, filed Mar. 28, 2001, the disclosures of which are incorporated by reference in their entirety. The aging process provides nanoparticle-oligonucleotide conjugates with unexpected enhanced stability and selectivity.

The method comprises providing oligonucleotides having covalently bound thereto a moiety comprising a functional group which can bind to the nanoparticles. The moieties and functional groups are those that allow for binding (i.e., by chemisorption or covalent bonding) of the oligonucleotides to nanoparticles. For instance, oligonucleotides having an alkanethiol, an alkanedisulfide or a cyclic disulfide covalently bound to their 5′ or 3′ ends can be used to bind the oligonucleotides to a variety of nanoparticles, including gold nanoparticles.

The oligonucleotides are contacted with the nanoparticles in water for a time sufficient to allow at least some of the oligonucleotides to bind to the nanoparticles by means of the functional groups. Such times can be determined empirically. For instance, it has been found that a time of about 12-24 hours gives good results. Other suitable conditions for binding of the oligonucleotides can also be determined empirically. For instance, a concentration of about 10-20 nM nanoparticles and incubation at room temperature gives good results.

Next, at least one salt is added to the water to form a salt solution. The salt can be any suitable water-soluble salt. For instance, the salt may be sodium chloride, magnesium chloride, potassium chloride, ammonium chloride, sodium acetate, ammonium acetate, a combination of two or more of these salts, or one of these salts in phosphate buffer. Advantageously, the salt is added as a concentrated solution, but it could be added as a solid. The salt can be added to the water all at one time or the salt is added gradually over time. By “gradually over time” is meant that the salt is added in at least two portions at intervals spaced apart by a period of time. Suitable time intervals can be determined empirically.

The ionic strength of the salt solution must be sufficient to overcome at least partially the electrostatic repulsion of the oligonucleotides from each other and, either the electrostatic attraction of the negatively-charged oligonucleotides for positively-charged nanoparticles, or the electrostatic repulsion of the negatively-charged oligonucleotides from negatively-charged nanoparticles. Gradually reducing the electrostatic attraction and repulsion by adding the salt gradually over time has been found to give the highest surface density of oligonucleotides on the nanoparticles. Suitable ionic strengths can be determined empirically for each salt or combination of salts. A final concentration of sodium chloride of from about 0.1 M to about 1.0 M in phosphate buffer, advantageously with the concentration of sodium chloride being increased gradually over time, has been found to give good results.

After adding the salt, the oligonucleotides and nanoparticles are incubated in the salt solution for an additional period of time sufficient to allow sufficient additional oligonucleotides to bind to the nanoparticles to produce the stable nanoparticle-oligonucleotide conjugates. As will be described in detail below, an increased surface density of the oligonucleotides on the nanoparticles has been found to stabilize the conjugates. The time of this incubation can be determined empirically. A total incubation time of about 24-48, particularly 40 hours, has been found to give good results (this is the total time of incubation; as noted above, the salt concentration can be increased gradually over this total time). This second period of incubation in the salt solution is referred to herein as the “aging” step. Other suitable conditions for this “aging” step can also be determined empirically. For instance, incubation at room temperature and pH 7.0 gives good results.

The conjugates produced by use of the “aging” step have been found to be considerably more stable than those produced without the “aging” step. As noted above, this increased stability is due to the increased density of the oligonucleotides on the surfaces of the nanoparticles which is achieved by the “aging” step. The surface density achieved by the “aging” step will depend on the size and type of nanoparticles and on the length, sequence and concentration of the oligonucleotides. A surface density adequate to make the nanoparticles stable and the conditions necessary to obtain it for a desired combination of nanoparticles and oligonucleotides can be determined empirically. Generally, a surface density of at least 10 picomoles/cm² will be adequate to provide stable nanoparticle-oligonucleotide conjugates. Particularly, the surface density is at least 15 picomoles/cm². Since the ability of the oligonucleotides of the conjugates to hybridize with nucleic acid and oligonucleotide targets can be diminished if the surface density is too great, the surface density is advantageously no greater than about 35-40 picomoles/cm².

As used herein, “stable” means that, for a period of at least six months after the conjugates are made, a majority of the oligonucleotides remain attached to the nanoparticles and the oligonucleotides are able to hybridize with nucleic acid and oligonucleotide targets under standard conditions encountered in methods of detecting nucleic acid and methods of nanofabrication.

It has been found that the hybridization efficiency of nanoparticle-oligonucleotide conjugates can be increased dramatically by the use of recognition oligonucleotides which comprise a recognition portion and a spacer portion. “Recognition oligonucleotides” are oligonucleotides which comprise a sequence complementary to at least a portion of the sequence of a nucleic acid or oligonucleotide target. In this embodiment, the recognition oligonucleotides comprise a recognition portion and a spacer portion, and it is the recognition portion which hybridizes to the linker oligonucleotides or the oligonucleotides attached to the other type of nanoparticles. The spacer portion of the recognition oligonucleotide is designed so that it can bind to the nanoparticles. For instance, the spacer portion could have a moiety covalently bound to it, the moiety comprising a functional group which can bind to the nanoparticles. These are the same moieties and functional groups as described above. As a result of the binding of the spacer portion of the recognition oligonucleotide to the nanoparticles, the recognition portion is spaced away from the surface of the nanoparticles and is more accessible for hybridization with its target. The length and sequence of the spacer portion providing good spacing of the recognition portion away from the nanoparticles can be determined empirically. It has been found that a spacer portion comprising at least about 10 nucleotides, particularly 10-30 nucleotides, gives good results. The spacer portion may have any sequence which does not interfere with the ability of the recognition oligonucleotides to become bound to the nanoparticles or to a nucleic acid or oligonucleotide target. For instance, the spacer portions should not have sequences complementary to each other, to that of the recognition oligonucleotides, to the linker oligonucleotide, or to the oligonucleotides attached to the other type of nanoparticles. Advantageously, the bases of the nucleotides of the spacer portion are all adenines, all thymines, all cytidines, or all guanines, unless this would cause one of the problems just mentioned. More suitably for the current invention, the bases are all adenines or all thymines. Most suitably the bases are all thymines.

It has further been found that the use of diluent oligonucleotides in addition to recognition oligonucleotides provides a means of tailoring the conjugates to give a desired level of hybridization. The diluent and recognition oligonucleotides have been found to attach to the nanoparticles in about the same proportion as their ratio in the solution contacted with the nanoparticles to prepare the conjugates. Thus, the ratio of the diluent to recognition oligonucleotides bound to the nanoparticles can be controlled so that the conjugates will participate in a desired number of hybridization events. The diluent oligonucleotides may have any sequence which does not interfere with the ability of the recognition oligonucleotides to be bound to the nanoparticles or to bind to the linker oligonucleotides or oligonucleotides attached to the other type of nanoparticles. The diluent oligonucleotides are also advantageously of a length shorter than that of the recognition oligonucleotides so that the recognition oligonucleotides can bind to their nucleic acid or oligonucleotide targets. If the recognition oligonucleotides comprise spacer portions, the diluent oligonucleotides are, most suitably, about the same length as the spacer portions. In this manner, the diluent oligonucleotides do not interfere with the ability of the recognition portions of the recognition oligonucleotides to hybridize with nucleic acid or oligonucleotide targets. Even more suitably, the diluent oligonucleotides have the same sequence as the sequence of the spacer portions of the recognition oligonucleotides.

### Preparation of Nanoparticle Aggregates

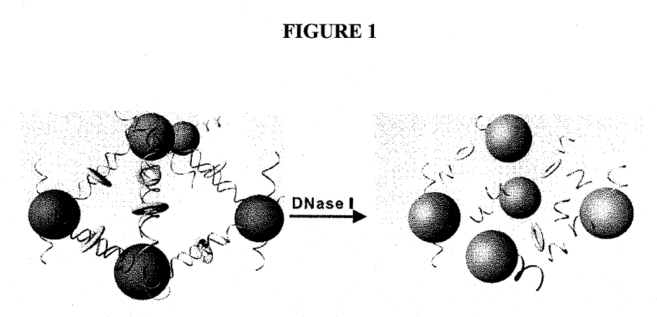

The nanoparticle aggregate can be prepared by allowing two types of nanoparticles having complementary oligonucleotides (a and a′) attached thereto to hybridize to form an aggregate (illustrated in FIG. 1). Since each type of nanoparticles has a plurality of oligonucleotides attached to it, each type of nanoparticles will hybridize to a plurality of the other type of nanoparticles. Thus, an aggregate is formed containing numerous nanoparticles of both types.

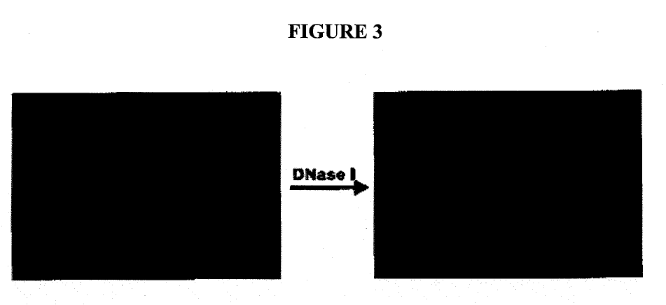

The nanoparticle aggregate can also be prepared by allowing two types of nanoparticles each having a plurality of oligonucleotides (either a or b) attached thereto, and each type of oligonucleotide can hybridize to a linker oligonucleotide. The linker oligonucleotide has a sequence comprising at least two portions a′ and b′, to which the oligonucleotides a and b is complementary, respectively. In a particular embodiment, the oligonucleotides a and b are functionalized to two types of gold nanoparticles in a way that oligonucleotide a is attached to the nanoparticle by its 3′ OH group, and oligonucleotide b is attached to the nanoparticle by the 5′ PO₄³⁻ group. See FIG. 3. After the oligonucleotides a and b are hybridized to the linker oligonucleotide, the 3′ OH group of oligonucleotide a is brought to close proximity to, and optionally can be ligated by a DNA ligase with, the 5′ PO₄³⁻ group of oligonucleotide b. The resulting double-stranded oligonucleotide, with or without ligation, constitutes a substrate for an endonuclease. A DNA ligase catalyzes the reaction joining the 5′ phosphate group of one nucleic acid molecule with the 3′ hydroxyl group of the other nucleic acid molecule. A suitable ligase for use in the current invention includes, but is not limited to, T4 DNA ligase. Those skilled in the art will appreciate that any ligase or enzyme capable of forming a phosphodiester bond between the 3′ OH group of oligonucleotide a and the 5′ PO₄³⁻ group of oligonucleotide b after hybridization to the linker oligonucleotide comprising at least two portions a′ and b′ is suitable for use in the current invention.

The at least two types of nanoparticles functionalized with oligonucleotides and/or linker oligonucleotides are hybridized to each other under conditions effective for hybridization of the oligonucleotides to form a nanoparticle aggregate. These hybridization conditions are well known in the art and can readily be optimized for the particular system employed. See, e.g., Sambrook et al., Molecular Cloning: A Laboratory Manual (2nd ed. 1989).

Faster hybridization can be obtained by freezing and thawing a solution containing the nucleic acid to be detected and the nanoparticle-oligonucleotide conjugates. The solution may be frozen in any convenient manner, such as placing it in a dry ice-alcohol bath for a sufficient time for the solution to freeze (generally about 1 minute for 100 μL of solution). The solution must be thawed at a temperature below the thermal denaturation temperature, which can conveniently be room temperature for most combinations of nanoparticle-oligonucleotide conjugates and free oligonucleotides. The hybridization is complete, the nanoparticle aggregate is formed, and the detectable change may be observed, after thawing the solution.

The rate of hybridization can also be increased by warming the solution containing the linker oligonucleotide and the nanoparticle-oligonucleotide conjugates to a temperature below the dissociation temperature (Tm) for the complex formed between the oligonucleotides on the nanoparticles and/or the linker oligonucleotides. Alternatively, rapid hybridization can be achieved by heating above the dissociation temperature (Tm) and allowing the solution to cool.

The rate of hybridization can also be increased by increasing the salt concentration (e.g., from 0.1 M to 0.3 M NaCl).

### Observation of Detectable Changes in the Method of Current Invention

The formation of gold nanoparticle aggregate and the dissociation of the aggregate result in an observable detectable change. The detectable change can be a color change, detectable by naked eyes. Alternatively, the change can be detected spectrophotometrically by measuring the extinction of a reaction mixture. See, A. A. Lazarides and G. C. Schatz, J. Phys. Chem. B., 104(3): 460-467 (2000).

The detectable change as a result of dissociation of the nanoparticle aggregate by endonuclease activity can be a color change. The formation of gold nanoparticle aggregate gives rise to a bluish purple color. Individual, non-aggregated nanoparticles display a red color when measuring the intensity of the surface plasmon band at about 520 nm. The color change occurs as a result of the formation of nanoparticles aggregate, which leads to a shift in the surface plasmon resonance of the nanoparticles. The dissociation of the aggregate and release of individual nanoparticles would change the color from bluish purple to red. The color changes can be observed with the naked eye or can be detected spectrophotometrically. Particularly advantageous is a color change observable with the naked eye, which is especially advantageous in a high throughput screening format.

The observation of a color change with the naked eye can be made more readily against a background of a contrasting color. For instance, when gold nanoparticles aggregates are used, the observation of a color change is facilitated by holding the sample against a white background, and the color change can be observed in solution. Alternatively, the observation of a color change is facilitated by spotting a sample of the reaction solution on a solid white surface (such as silica or alumina TLC plates, filter paper, cellulose nitrate membranes, and nylon membranes, particularly a C-18 silica TLC plate) and allowing the spot to dry. Initially, the spot retains the color of the hybridization solution (which ranges from pink/red, in the absence of hybridization, to purplish-red/purple, if there has been hybridization). On drying at room temperature or 80° C. (temperature is not critical), a blue spot develops if the nanoparticles remain as aggregate. In the presence of an endonuclease activity, the spot is pink, because the nanoparticle aggregate is released to individual gold nanoparticles. The blue and the pink spots are stable and do not change on subsequent cooling or heating or over time. They provide a convenient permanent record of the test. No other steps (such as a separation of individual or aggregated nanoparticles) are necessary to observe the color change.

In one embodiment, the color change is detected by measuring and comparing light absorbance of the endonuclease reaction mixture containing a test compound in a spectrophotometer relative to the light absorbance of a control reaction mixture having no compound. Advantageously, the detectable change is measured at wavelength of 520 nm.

Gold nanoparticle suitable for use in the current invention has a diameter ranging from about 5 nm to about 250 nm. In a further embodiment, the gold nanoparticle has a diameter of 13 nm. Different gold nanoparticle aggregates each made of nanoparticles of different sizes can be measured at different wavelength.

Nuclease as used herein refers to an enzyme that cleaves the phosphodiester bonds between the nucleotide subunits of a DNA or RNA molecule. Nucleases can be categorized as endonucleases or exonucleases. Endonucleases can be further categorized as sequence-specific or non sequence-specific endonuclease. Examples of sequence-specific endonucleases include bacteria restriction enzymes that recognize and cleave a specific sequence in double-stranded DNA. Non sequence-specific endonucleases such as DNase I does not recognize a specific sequence as substrate.

In one embodiment, the invention provides a method for screening a compound for its effect on a sequence-specific endonuclease. The oligonucleotides for use in this embodiment are designed in a way that when the oligonucleotides hybridized to each other (as in the embodiment where the aggregate consists of two types of nanoparticles) or to one another (as in the embodiment where the aggregate is formed by two types of nanoparticles and a linker oligonucleotide), the double-stranded oligonucleotides comprises the recognition sequence of the endonuclease.

In another embodiment, the invention provides a method for screening a compound for its effect on a non sequence-specific endonuclease. The oligonucleotides for use in this embodiment can be, in principle, any complementary oligonucleotides.

The invention is particularly applicable wherein the endonuclease is a bacterial endonuclease, a fungal endonuclease, or a viral endonuclease.

In one aspect, the invention provides a method for detecting an endonuclease inhibitor in a sample. In another aspect, the invention provides a method for detecting or measuring endonuclease activity in a sample.

The instant invention can be easily adapted to high-throughput screening methods, which can be used to determine potential endonuclease inhibitors. Particularly, the method can be used to rapidly identify, from a large numbers of compounds in the compound libraries, potential anti-microbial and anti-viral agents in combinatorial formats.

In one embodiment of the invention, the method further comprises contacting the gold nanoparticle aggregate substrate and the endonuclease in the presence or absence of the plurality of compounds to form a plurality of reaction mixtures and incubating the plurality of reaction mixtures under conditions sufficient to allow the endonuclease reaction to occur; and determining the effect of at least one of the plurality of compounds on the endonuclease activity by observing a detectable change between the reaction mixtures in the presence of the plurality of compounds and the reaction mixtures in the absence of the plurality of compounds, wherein a detectable change indicates that the compound has an effect on the endonuclease activity.

### Kit for the Current Invention

The invention also provides kits for screening a compound for its effect on endonuclease activity. In one embodiment, the kit comprises at least two containers, the first container holding a composition of gold nanoparticle aggregate in a buffer solution, and the second container holding an endonuclease enzyme. In another embodiment, the kit comprises at least three containers, the first container holding gold nanoparticle aggregates, the second container holding an endonuclease enzyme, and the third container holding a buffer. In a further embodiment, the container holds a lyophilized endonuclease enzyme.

In another aspect, the invention also provides kits for detecting an endonuclease activity or for measuring an endonuclease activity in a sample. In yet another aspect, the invention provides kits for detecting an endonuclease inhibitor in a sample.

It is to be noted that the term “a” or “an” entity refers to one or more of that entity. For example, “a characteristic” refers to one or more characteristics or at least one characteristic. As such, the terms “a” (or “an”), “one or more” and “at least one” are used interchangeably herein. It is also to be noted that the terms “comprising”, “including”, and “having” have been used interchangeably.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the claims.

### EXAMPLES

### Example 1

### Preparation of Nanoparticle Aggregate Substrate without the Linker Oligonucleotide and Calorimetric Screening Assay

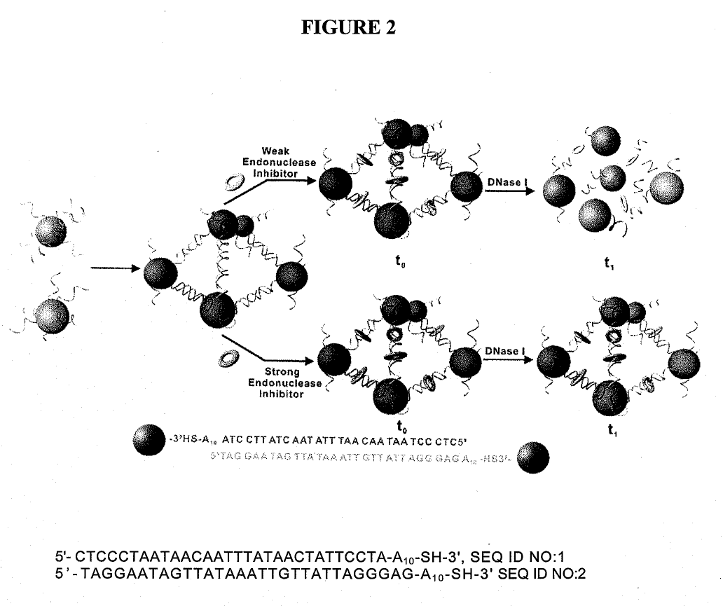

Nanoparticles were prepared by functionalizing two separate batches of 13 nm gold nanoparticles with two different thiol-modified oligonucleotide strands DNA-1 and DNA-2, respectively. These particles are denoted AuNP-1 and AuNP-2 (DNA-1: 5′-CTCCCTAATAACAATTTATAACTATTCCTA-A₁₀-SH-3′, SEQ ID NO:1; DNA-2: 5′-TAGGAATAGTTATAAATTGTTATTAGGGAG-A₁₀-SH-3′ SEQ ID NO:2). DNA-1 and DNA-2 are complementary to each other. Therefore, AuNP-1 and AuNP-2 can hybridize and form a cross-linked network of nanoparticles, which appears purple in color due to the red-shifted plasmon band of the gold nanoparticles (13 nm). This red-shifting is a well-understood process and a highly diagnostic feature of aggregate formation. (See N. L. Rosi, D. A. Mirkin, Chem. Rev., 105: 1047 (2005)). (See also U.S. Pat. No. 6,506,564, the disclosure of which is incorporated herein by reference in its entirety). These aggregates can then be used as colorimetric indicators of endonuclease activity. As the endonuclease cleaves the DNA duplex interconnects, particles are released regenerating a red color due to the dispersed nanoparticles. The color can be observed with the naked eye, or the extinction (or optical density or optical absorbance) can be measured by UV-vis spectroscopy at 520 nm. (FIG. 2)

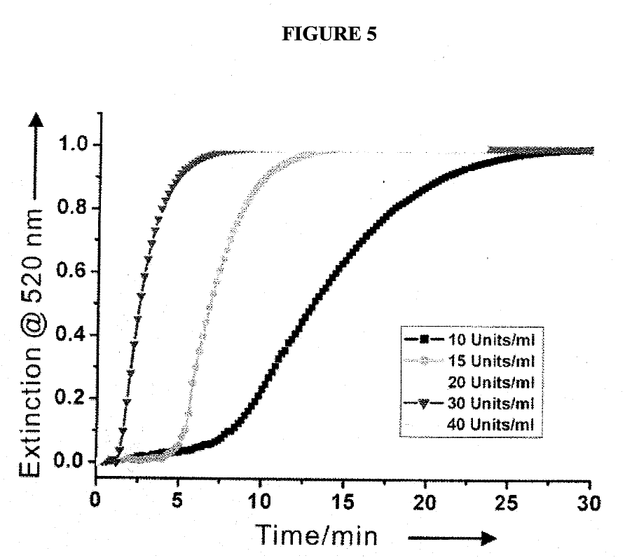

The aforementioned aggregates were used to evaluate the enzymatic activity of DNase I. In a typical experiment, DNase I, at predetermined concentrations, was added to a solution of the aggregates (10 units/ml, 15 units/ml, 20 units/ml, 30 units/ml and 40 units/ml). The color of the solution gradually changed from purple to red. By measuring the absorbance at 520 nm, we could quantitatively follow the nucleic acid hydrolysis process catalyzed by DNase I (FIG. 5). The reaction rate increases with increasing enzyme concentration and can be followed in real time.

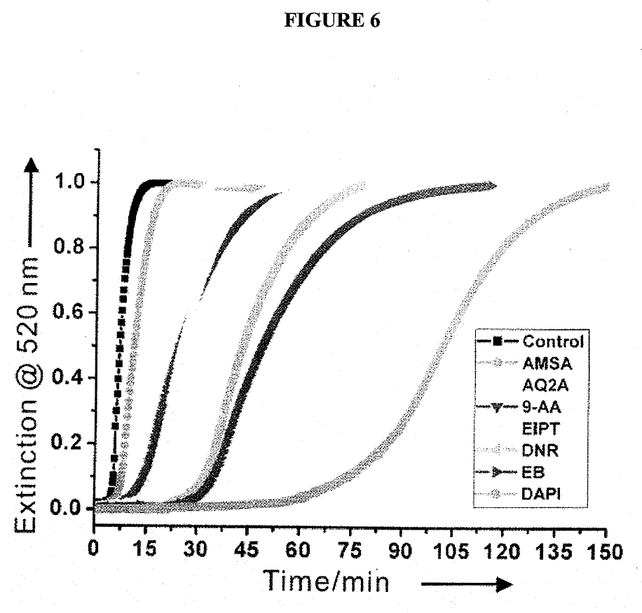

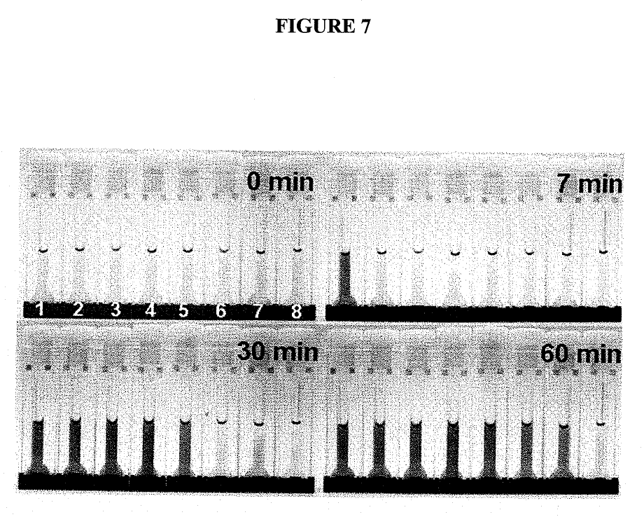

In addition to screening enzyme activity, one can easily use the assay to evaluate the efficiency of inhibitors of DNase I. In a typical screening experiment, DNase I (15 units/ml) was added to solutions of the DNA-linked gold nanoparticle aggregates in the presence of one of the following DNA binding molecules: amsacrine (AMSA), anthraquinone-2-carboxylic acid (AQ2A), 9-aminoacridine (9-AA), ellipticine (EIPT), daunorubicin (DNR), ethidium bromide (EB) and 4′,6-diamidedino-2-phenylindole (DAPI) (1 μM), respectively. These DNA binding molecules are known to inhibit DNase I. (See R. A. Ikeda, P. B. Dervan, J. Am. Chem. Soc., 104: 296 (1982); S. M. Forrow, M. Lee, R. L. Souhami, J. A. Hartley, Chem. Biol. Interact., 96: 125 (1995); P. G. Baraldi, A. Bovero, F. Fruttarolo, D. Preti, M. A. Tabrizi, M. G. Pavani, R. Romagnoli, Med. Res. Rev., 24: 475 (2004)). Extinction was monitored at 520 nm as a function of time (sample scan rate=5 min⁻), and the color of the solution was followed with the naked eye. The time at which 50% of the aggregate is hydrolyzed (T_{H}) can be used as a measure of inhibition (FIG. 6). The inhibitors decrease DNase I activity and increase the T_{H} and, therefore, the corresponding time required for a color change in the solution. The inhibitors studied exhibit a trend of inhibition potency as follows: DAPI>EB>DNR>9-AA, EIPT, and AQ2A>AMSA. The trend is determined based on T_{H}, which is consistent with the relative binding affinities of the inhibitors to DNA, as determined by measuring the melting temperature of the duplex DNA in the presence of each DNA-binding molecule (FIG. 6 and Table 1 below). This approach can be used for the high throughput screening of endonuclease inhibitors through visual inspection as demonstrated in FIG. 7, and the relative degree of endonuclease inhibition can be differentiated easily.

Table 1 below shows melting temperatures (Tm) of duplex DNA (without nanoparticles) in the presence of a specific endonuclease inhibitor (left column). The relative binding affinities of the inhibitors to DNA are consistent with the hydrolysis time (T_{H}) of the aggregates by DNase I in the presence of a specific inhibitor. T_{H} is determined by measuring the extinction at 520 nm and determining when half of the particles composing the aggregates have been released.

TABLE 1

| Endonuclease Inhibitors | Tm^{[a]} [° C.] | T_{H} [min] |
|---|---|---|
| Control | 61.0 | 6.8 |
| AMSA | 61.5 | 11.0 |
| AQ2A | 62.5 | 19.6 |
| 9-AA | 62.5 | 24.6 |
| EIPT | 63.0 | 25.2 |
| DNR | 65.5 | 43.2 |
| EB | 65.5 | 48.8 |
| DAPI | 77.0 | 101.2 |

^{[a]}Condition: DNA duplex (2.0 μM) in sodium phosphate buffer (10 mM, pH 7.0) containing sodium chloride (100 mM) in the presence of DNA binding molecule (5.0 μM)

In conclusion, we have developed a new colorimetric assay for screening endonuclease activity and determining the relative inhibitory potencies of potential inhibitors by monitoring the kinetics of DNA-AuNP aggregate dissociation. Compared to other assays, this screening approach is simpler, easy to monitor, and provides a rapid qualitative indication of relative inhibition capabilities. For high-throughput screening processes, a way of qualitatively measuring differences in endonuclease activity is extremely useful. (See J. Bajorath, Nat. Rev. Drug Discovery, 1: 882 (2002)).

### Experimental Procedure

AuNP-1 and AuNP-2 were functionalized with 3′-thiol-modified 30-mer oligonucleotides (AuNP-1, DNA-1 sequence: 5′-CTCCCTAATAACAATTTATAACTATTCCTA-A₁₀-SH-3′, SEQ ID NO: 1; AuNP-2, DNA-2 sequence: 5′-TAGGAATAGTTATAAATTGTTATTAGGGAG-A₁₀-SH-3′, SEQ ID NO:2) using previously reported methods. (See J. J. Storhoff, R. Elghanian, R. C. Mucic, C. A. Mirkin, R. L. Letsinger, J. Am. Chem. Soc., 120: 1959 (1998)). After combining 1 ml aliquots of the two probes, AuNP-1 and AuNP-2 (each at 3 nM), in hybridization buffer (50 mM Tris-HCl buffer pH 7.0, 2 mM MgCl₂), the mixture was heated to 90° C. and held at this temperature for 10 minutes. The solution was cooled to room temperature, which resulted in hybridization of the particles and the concomitant diagnostic red to purple color change.

The pre-prepared gold nanoparticle aggregates were washed (3 times) with DNase I buffer (10 mM sodium phosphate buffer pH 7.0, 0.75 mM MgCl₂). They were then resuspended in 1 ml of DNase I buffer. A 10 μL aliquot of each of the endonuclease inhibitors (0.1 mM) was then added to a 1 ml solution of the aggregates, respectively, and incubated for 10 minutes. The assay was initiated by adding DNase I, and the hydrolysis kinetics were monitored by UV-vis spectroscopy (Cary 5000, Varian). The solution was continuously stirred with a magnetic stir bar at room temperature to keep the aggregates suspended.

### Example 2

### Preparation of Gold Nanoparticle Aggregate Substrate Including the Linker Oligonucleotide and Calorimetric Screening Assay Using the Gold Nanoparticle Aggregate Substrate

Gold nanoparticle substrates were prepared by functionalizing two separate batches of 13 nm gold particles with two different thiol-modified oligonucleotide strands DNA-3 and DNA-4. Those particles are denoted as AaNP-3 and AuNP-4 (DNA-3: 5′-SH-A₁₀-CGTTAGGACTTACGC-OH-3′, SEQ ID NO:3; DNA-4: 5′-PO₄³⁻-TTATAACTATTCCTA-A₁₀-SH-3′, SEQ ID NO:4). DNA-3 and DNA-4 are both half-complementary to a 30-mer oligonucleotide (DNA-linker sequence; 5′ TAGGAATAGTTATAAGCGTAAGTCCTAACG-3′, SEQ ID NO:5). Therefore, AuNP 3 and AuNP-4 can cross-link to each other by hybridization with the 30-mer oligonucleotide linker followed by ligation with T4 ligase, which converts the three-strand 30-bp nicked structure to a continuous two-strand 30-bp duplex. (See A. G. Kanaras, Wang, A. D. Bates, R. Cosstick, M. Burst, Angew Chem. Int. Ed., 42: 191 (2003); X. Xu. N. L. Rose, Y. Wang, F. Huo. C. A. Mirkin, J. Am. Chem. Soc., 128: 9286 (2006)). This polymerization process results in a purple-colored nanoparticle assembly. Note that the dispersed DNA-AuNPs of 13 nm diameter display a brilliant red color, which is due to the plasmon band of the particles centered at about 520 nm.

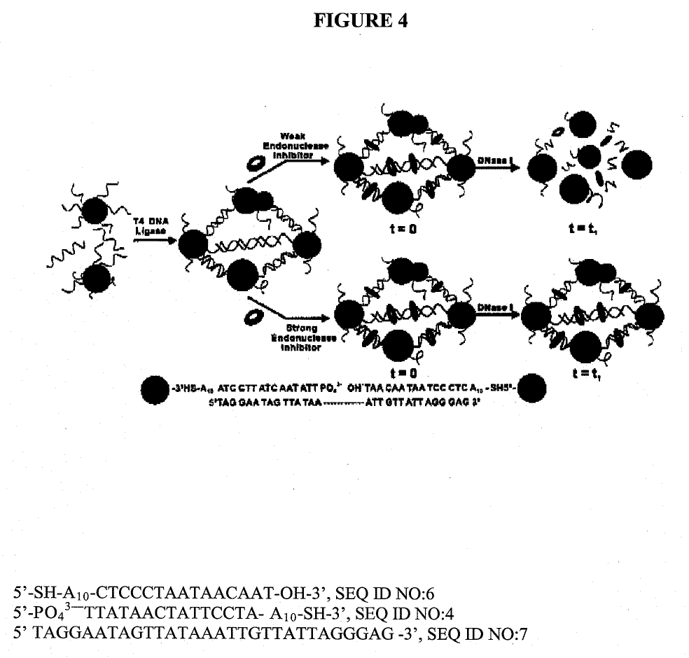

The system presented herein was designed such that the assemblies can be dissociated to release gold nanoparticles using a representative non-sequence specific endonuclease such as DNase I, which non-specifically cleaves duplex DNA. The process of aggregate dissociation results in a purple-to-red color change. The color change can be observed with the unassisted eye or a UV-vis spectroscopy. (FIG. 4). Gold nanoparticle aggregates with duplex interconnects were used to test the enzyme activity of DNase I. After addition of DNase I, the aggregate dissociates and disperses the gold nanoparticles. The color of the solution concomitantly changed from purple to red. By measuring the absorbance 520 nm, which is a diagnostic indicator of the dispersed AuNPs, we could quantitatively follow the hydrolysis process catalyzed by DNase I.

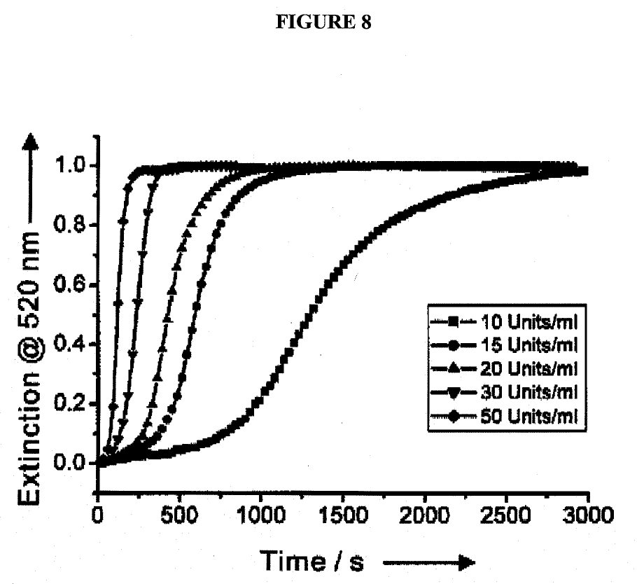

FIG. 8 shows that the substrate is operative in real time and the apparent reaction rate increases with increasing enzyme concentration. Note that we prepared another AuNP aggregate as shown in Example 1 by simply mixing two sets of AuNPs which were each functionalized with a plurality of complementary thiol-modified oligonucleotide strands. However, the ligated aggregates are more sensitive to endonuclease activity for several reasons: there are fewer interparticle duplexes due to the lower hybridization efficiency of the three-strand DNA hybridization system; and the yield of DNA ligation within the gold nanoparticle aggregate by T4 ligase is relatively low due to steric hindrance. Also, the ligated materials are less sensitive to buffer conditions such as pH and salt concentration.

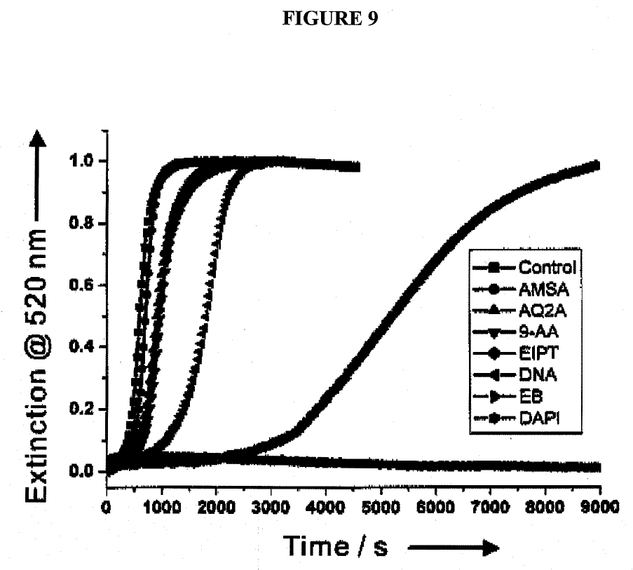

The assay method can be used for screening inhibitors of DNase I. This has been demonstrated by using DNA-binding molecules, which are potent DNase I inhibitors. A typical screening experiment was slated by adding the DNase I to solutions of the gold nanoparticle aggregates in the presence of one of the DNA binding molecules, amsacrine (AMSA), anthrquinone-2-carboxylic acid (AQ2A), 9-aminoacridine (9-AA), ellipticine (EIPT), daunorubicin (DNR), ethidium bromide (EB) and 4′,6-diamidedino-2-phenylindole (DAPI) (1 μM), respectively. The color changes of the solutions were monitored at 520 nm by UV-vis spectrometry over time (sample scan rate=2 min⁻¹). The time at which 50% of the aggregate is hydrolyzed can be determined by the kinetic curve as shown in FIG. 9. The inhibitors can decrease DNase I activity and increase the hydrolysis time and the corresponding time required for a color change in the solution. Thus, we could determine the relative activities of different endonuclease inhibitors by measuring the time required to hydrolyze 50% of the aggregates (t). Based on the results shown in FIG. 9, the inhibitory potency of the inhibitors exhibits the following trend: DAPI>EB>DNR>9-AA, EIPT, and AQ2A>AMSA according to hydrolysis time. The hydrolysis time is consistent with relative binding affinities of inhibitors to DNA by determining the melting temperature (Tm) of duplex DNA at 260 nm in the presence of each DNA-binding molecule. Table 2 below shows melting temperature (Tm) of the duplex DNA (without nanoparticles) and releasing time (t) of the gold nanoparticle substrates in the presence of endonuclease inhibitors.

TABLE 2

| Endonuclease Inhibitors | Tm^{[a]} [° C.] | t [min] |
|---|---|---|
| Control | 61.0 | 9.5 |
| AMSA | 61.5 | 11.5 |
| AQ2A | 62.5 | 14.5 |
| 9-AA | 62.5 | 16.0 |
| EIPT | 63.0 | 15.0 |
| DNR | 65.5 | 31.5 |
| EB | 65.5 | 82.0 |
| DAPI | 77.0 | >1440 |

^{[a]}Condition: DNA duplex (2.0 μM) in sodium phosphate buffer (10 mM, pH 7.0) containing sodium chloride (100 mM) in the presence of DNA binding molecule (5.0 μM)

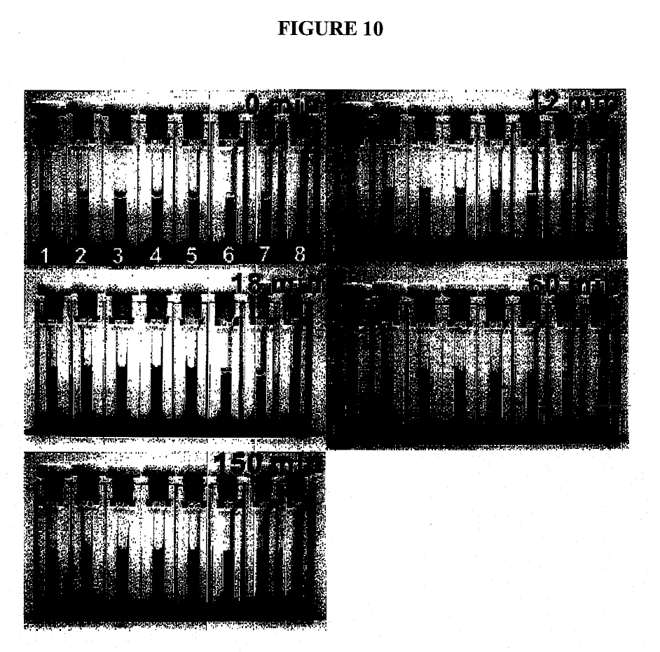

High-throughput screening is widely used for the identification of hit compounds from combinatorial libraries. (See P. A. Johnston, P. A. Johnston, Drug Discovery Today, 7: 353 (2002); D. L. Boger, J. Deshamais, K. Capps, Angew. Chem. Int. Ed., 42: 4138 (2003); S. Wang, T. B. Sim, Y.-S. Kim, Y.-T. Chang, Cum Opin. Cham. Biol., 8: 371 (2004)). We have examined the application of the gold nanoparticle aggregates for high throughput screening of endonuclease inhibitors by visual inspection. The catalytic hydrolysis of gold nanoparticle aggregates with DNA duplex interconnects by endonucleases accompanies a color change from purple to red: no color change or slow color change hence indicates inhibition of endonuclease activity. Thus, we can determine potencies of inhibitor using color change as a function of time. As shown in FIG. 10, all eight cells (one control and seven containing DNA-binding molecules) appear light purple at t=0 and the color of the cells changed from light purple to red as a function of reaction time except DAPI, which remains light purple throughout the examination time. These results show the discrimination between weak, intermediate, and strong endonuclease inhibitors by an easily identified color change. The trend of potencies of inhibitors was determined to be DAPI>EB>DNR>9-AA, EIPT, and AQ2A>AMSA, which is consistent with the control experiments involving serial analysis of each inhibitors with nanoparticle-free duplex DNA.

In conclusion, we have developed a new high-throughput colorimetric assay for screening endonuclease activities and determining the relative inhibitory potencies of endonuclease inhibitors by monitoring the kinetics of DNA-AuNP aggregate dissociation.

Compared to other assays, this screening approach is simpler, easy to monitor, and provides both qualitative and quantitative indication of inhibition. This assay can be easily adapted to high-throughput screening methods, which can be used to determine potential anti-microbial and anti-virus agents from large combinatorial libraries and screen endonuclease activities from large biocatalyst libraries.

### Experimental Procedure

Two separate types of DNA-AuNPs were prepared by functionalizing 13 nm gold nanoparticles with two different thiol-modified oligonucleotide strands, AuNP-3 and AuNP-4. AuNP-3 was functionalized with 3′-hydroxyl-modified and 5′-thiol-modified 15-mer oligonucleotides (DNA-3 sequence: 5′-SH-A₁₀-CGTTAGGACTTACGC-OH-3′, SEQ ID NO:3). DNA-3 is complementary to one-half of the 30-mer linker oligonucleotide (DNA-linker oligonucleotide sequence: 5′ TAGGAATAGTTATAAGCGTAAGTCCTAACG-3′, SEQ ID NO:5). AuNP-4 was functionalized with 3′-thiolated and 5′-phosphorylated 15-mer oligonucleotides (DNA-4: 5′-PO₄³⁻-TTATAACTATTCCTA-A₁₀-SH-3′, SEQ ID NO:4) that are complementary to the other half of the linker-oligonucleotide. After combining the three components, AuNP-3 and AuNP-4 (each at 3 nM) as well as the linker-oligonucleotides (3 μM), in the presence of ligation buffer (50 mM Tris-HCl buffer pH 7.5, 5 mM MgCl₂, 1 mmol ATP, 0.05 mg/ml BSA, T4 DNA Ligase 4.000 units/ml), the DNAs assemble to form a three-strand DNA hybridization complex. First the linker-oligonucleotide acts as templates to co-align the 3′-hydroxyl group of the DNA-3 and the 5′-phosphate group of the DNA-4. Then the T4 DNA Ligase catalyzes the formation of a phosphodiester bond between the 3′-hydroxyl and the 5′-phosphate groups of DNA-3 and DNA-4, covalently joining DNA-3 and DNA-4 to form a 30-mer oligonucleotide. This method yield stable 30-base-pair double stranded links between the AuNPs. Since the nanoparticles aggregate, a red-to-purple color change can be observed.

The prepared gold nanoparticle aggregate was washed (3 times) with DNase I buffer (10 mM sodium phosphate buffer pH 7.0, 0.25 mM MgCl₂). The assay was initiated by adding DNase I (10-50 units/ml) and the hydrolysis kinetics were monitored by a UV-vis spectrum at the extinction of 520 nm with stirring at room temperature.

What is claimed is:

1. A method for screening one or more compounds for its effect on an activity of one or more endonucleases, said method comprising steps of:
contacting one or more types of gold nanoparticle aggregate substrates with one or more types of endonucleases, in the presence of absence of one or more types of compounds to form at least one reaction mixture and incubating the reaction mixture under conditions sufficient to allow the endonuclease reaction to occur, wherein the gold nanoparticle aggregate substrate comprises at least two types of gold nanoparticles, the first type of gold nanoparticle having one or more first oligonucleotides bound thereto, and the second type of gold nanoparticle having one or more second oligonucleotides bound thereto, the first oligonucleotide and the second oligonucleotide having sequences that are at least partially complementary to each other, wherein the gold nanoparticle aggregate is formed by hybridization of the first and second oligonucleotides, and each type of gold nanoparticle aggregate substrate comprises gold nanoparticles of a unique particle size; and
determining the effect of the compound on the endonuclease activity by observing a detectable change between the reaction mixture in the presence of the compound and the reaction mixture in the absence of the compound, wherein a detectable change indicates that the compound has an effect on the endonuclease activity.

2. The method of claim 1, wherein the endonuclease is a sequence specific endonuclease or a non-sequence specific endonuclease.

3. The method of claim 1, wherein the gold nanoparticle aggregate substrate, the endonuclease, and the compound are added simultaneously to form a reaction mixture.

4. The method of claim 1, wherein the gold nanoparticle aggregate substrate is contacted with the compound to form a first reaction mixture, and the endonuclease is contacted with the first reaction mixture to form a second reaction mixture, and wherein the second reaction mixture is incubated under conditions sufficient to allow the endonuclease reaction to occur.

5. The method of claim 1, wherein the endonuclease is contacted with the compound to form a first reaction mixture, and the gold nanoparticle aggregate is contacted with the first reaction mixture to form a second reaction mixture, and wherein the second reaction mixture is incubated under conditions sufficient to allow the endonuclease reaction to occur.

6. The method of claim 1, wherein the gold nanoparticle has a diameter ranging from about 5 nm to about 250 nm.

7. The method of claim 6, wherein the gold nanoparticle has a diameter of 13 nm.

8. A method for screening one or more compounds for their effect on an activity of one or more endonucleases, said method comprising steps of:
contacting one or more gold nanoparticle aggregate substrates and one or more endonuclease in the presence or absence of one or more compounds to form at least one reaction mixture and incubating the reaction mixture under conditions sufficient to allow the endonuclease reaction to occur, wherein the gold nanoparticle aggregate substrate comprises (1) at least two types of gold nanoparticles, the first type of gold nanoparticle having one or more first oligonucleotides bound thereto, and the second type of gold nanoparticle having one or more second oligonucleotides bound thereto, and (2) at least one type of linker oligonucleotide, the first oligonucleotide and the second oligonucleotide having sequences that are at least partially complementary to the sequence of the linker oligonucleotide, and wherein the gold nanoparticle aggregate is formed by hybridization of the first and second oligonucleotides to the linker oligonucleotide and each type of gold nanoparticle aggregate substrate comprises gold nanoparticles of a unique particle size; and
determining the effect of the compound on the endonuclease activity by observing a detectable change between the reaction mixture in the presence of the compound and the reaction mixture in the absence of the compound, wherein a detectable change indicates that the compound has an effect on the endonuclease activity.

9. The method of claim 8, wherein the endonuclease is a sequence specific endonuclease or a non-sequence specific endonuclease.

10. The method of claim 8, wherein the gold nanoparticle aggregate substrate, the endonuclease, and the compound are added simultaneously to form a reaction mixture.

11. The method of claim 8, wherein the gold nanoparticle aggregate substrate is contacted with the compound to form a first reaction mixture, and the endonuclease is contacted with the first reaction mixture to form a second reaction mixture, and wherein the second reaction mixture is incubated under conditions sufficient to allow the endonuclease reaction to occur.

12. The method of claim 8, wherein the endonuclease is contacted with the compound to form a first reaction mixture, and the gold nanoparticle aggregate is contacted with the first reaction mixture to form a second reaction mixture, and wherein the second reaction mixture is incubated under conditions sufficient to allow the endonuclease reaction to occur.

13. The method of claim 8, wherein the gold nanoparticle has a diameter ranging from about 5 nm to about 250 nm.

14. The method of claim 13, wherein the gold nanoparticle has a diameter of 13 nm.

15. The method of claim 8, further comprising:
contacting the gold nanoparticle aggregate substrate and the endonuclease in the presence or absence of a plurality of compounds to form a plurality of reaction mixtures and incubating the plurality of reaction mixtures under conditions sufficient to allow the endonuclease reaction to occur; and
determining the effect of at least one of the plurality of compounds on the endonuclease activity by observing a detectable change between the reaction mixtures in the presence of the plurality of compounds and the reaction mixtures in the absence of the plurality of compounds, wherein a detectable change indicates that the compound has an effect on the endonuclease activity.

16. The method of claim 15, wherein the gold nanoparticle aggregate substrate, the endonuclease, and each of the plurality of compounds are added simultaneously to form a plurality of reaction mixtures.

17. The method of claim 15, wherein the gold nanoparticle aggregate substrate is contacted with each of the plurality of compounds to form a plurality of first reaction mixtures, and the endonuclease is contacted with each of the first reaction mixture to form a plurality of second reaction mixtures, and wherein each of the second reaction mixture is incubated under conditions sufficient to allow the endonuclease reaction to occur.

18. The method of claim 15, wherein the endonuclease is contacted with each of the plurality of compounds to form a plurality of first reaction mixtures, and the gold nanoparticle aggregate substrate is contacted with each of the first reaction mixture to form a plurality of second reaction mixtures, and wherein each of the second reaction mixture is incubated under conditions sufficient to allow the endonuclease reaction to occur.

19. A kit for screening for an endonuclease inhibitor comprising:
(a) a composition comprising a gold nanoparticle aggregate;
(b) an endonuclease enzyme;
(c) a buffer; and optionally
(d) an instruction manual.

20. The kit of claim 19, wherein the endonuclease enzyme is a lyophilized endonuclease enzyme.
